(19) United States
(12) Patent Application Publication
(10) Pub. No.: US 2025/0194936 A1
(43) Pub. Date: Jun. 19, 2025

(54) Smart Stent with Triboelectric Nano-Generators (TENG) to Detect Restenosis

(71) Applicant: Bard Peripheral Vascular, Inc., Franklin Lakes, NJ (US)

(72) Inventors: Rahim Akbar Ali Jindani, Phoenix, AZ (US); Nathan P. Retzlaff, Kenosha, WI (US); Brandon Simmons, Tempe, AZ (US)

(21) Appl. No.: 18/840,840

(22) PCT Filed: Feb. 24, 2022
(86) PCT No.: PCT/US22/17742

§ 371 Date: Aug. 22, 2024

(51) Int. Cl. A61B 5/02; A61B 5/00; A61B 5/026; A61B 5/07; A61F 2/07

(52) U.S. Cl. CPC A61B 5/02007; A61B 5/026; A61B 5/076; A61B 5/4851; A61B 5/6862; A61F 2/07; A61B 2562/0261

(57) ABSTRACT

Embodiments disclosed herein are directed to a prosthesis defining a lumen and including a graft disposed on an outer surface, and a triboelectric nano-generator (TENG) coupled to an inner surface thereof. The TENG can move relative to prosthesis in one of a rotational, radial, or linear movement. The TENG can be coupled to the prosthesis at the upstream and downstream ends to define an air gap therebetween. The TENG can be formed of a first material (e.g. polytetrafluoroethylene, PTFE), the graft can be formed of a second material (e.g. polycarbonate, nylon, LDPE, HOPE). The difference in charge affinity can create a voltage output as the TENG is moved by blood flowing through the lumen. The voltage output can be communicated to a console to determine blood flow information and determine the onset or rate of restenosis.

### BACKGROUND

Peripheral Arterial Disease (“PAD”) is one of the leading causes for severe mobility issues, pain, heart diseases and discomfort to patients. Worldwide 202 million people suffer from PAD. Many of these patients who suffer from PAD often require the use of intravascular prosthetics, such as stents and stent grafts to restore patency to a vessel, such as an artery, vein, or valve, etc., and improve their quality of life.

Restenosis can occur where there is a recurrence in the narrowing of the vessel after placement of the prosthesis. Restenosis can be common in patients but the causes are poorly understood. Restenosis can occur in 32%-55% of patients following the procedure, and can be due to various environmental factors, poor health choices or pre-existing conditions. Restenosis can occur between 6-12 months after the procedure and is often only detected when the condition has advanced to such an extent that the patient is once again suffering the effects of occluded vessels and PAD.

Currently there is a need to monitor the blood flow continuously through a graft or stent, which could help in the prognosis and understanding of the underlying causes of the condition. Such data can provide early detection of atherosclerotic lesion formation and restenosis, and can provide more data driven analysis for physicians. Embodiments disclosed herein are directed to address the foregoing.

### SUMMARY

Disclosed herein is a system for detecting restenosis of a vessel including, a prosthesis disposed within the vessel and defining a lumen, the prosthesis including, a graft disposed on an outer surface of the prosthesis, and a triboelectric nano-generator (TENG) coupled to an inner surface of the prosthesis, the TENG configured to move relative to the prosthesis to generate a voltage output, and a console communicatively coupled to the prosthesis and configured to receive and analyze the voltage output and determine a blood flow information through the lumen of the prosthesis.

In some embodiments, the TENG is rotatably coupled to the prosthesis and detects a change in blood flow through the lumen by a change in rotational movement of the TENG relative to the prosthesis.

In some embodiments, the TENG is slidably coupled to the prosthesis and detects a change in blood flow through the lumen by a change in radial outward and inward movement of the TENG relative to the prosthesis.

In some embodiments, the TENG is slidably coupled to the prosthesis and detects a change in blood flow through the lumen by a change in back and forth linear movement of the TENG, parallel to a direction of blood flow, relative to the prosthesis.

In some embodiments, the TENG includes a first material and the graft includes a second material, the first material includes one of polytetrafluoroethylene (PTFE), expanded polytetrafluoroethylene (ePTFE), or Kapton, and the second material includes one or more of polycarbonate, nylon, low density polyethylene (LDPE), or high density polyethylene (HDPE).

In some embodiments, the second material includes a difference in charge affinity (nC/J) of at least +/−40 nC/J relative to the first material.

In some embodiments, the prosthesis includes a frame formed of a third material including one of a plastic, polymer, nylon, metal, alloy, or Nitinol.

In some embodiments, the TENG includes a first material, the graft includes a second material, and the prosthesis further includes a frame formed of a third material, the first material, second material and third material being the same or different.

In some embodiments, the first material may be selected from one or more of a plastic, polymer, polycarbonate, nylon, low-density polyethylene (LDPE), high-density polyethylene (HDPE), polytetrafluoroethylene (PTFE), expanded polytetrafluoroethylene (ePTFE), polyimide films, and poly (4,4′-oxydiphenylene-pyromellitimide) (“Kapton”).

In some embodiments, the second material may be selected from one or more of a plastic, polymer, polycarbonate, nylon, low-density polyethylene (LDPE), high-density polyethylene (HDPE), polytetrafluoroethylene (PTFE), expanded polytetrafluoroethylene (ePTFE), polyimide films, poly (4,4′-oxydiphenylene-pyromellitimide) (“Kapton”), metal, alloy, composite, stainless steel, and Nitinol.

In some embodiments, the second material may be selected from one or more of a plastic, polymer, polycarbonate, nylon, low-density polyethylene (LDPE), high-density polyethylene (HDPE), polytetrafluoroethylene (PTFE), expanded polytetrafluoroethylene (ePTFE), polyimide films, and poly (4,4′-oxydiphenylene-pyromellitimide) (“Kapton”).

In some embodiments, the prosthesis is configured to transition between a first configuration having a first diameter and a second configuration having a second diameter, the second diameter being larger than the first diameter.

In some embodiments, the prosthesis further includes an antenna configured to communicatively couple the prosthesis to an external computing device.

In some embodiments, the console is disposed on the prosthesis and is configured to determine the blood flow information and communicate the blood flow information to the external computing device.

In some embodiments, the console is disposed on the external computing device and the antenna communicates voltage output from the TENG to the console to determine the blood flow information.

In some embodiments, the prosthesis further includes a blood flow sensor configured to receive a voltage output from the TENG to power the blood flow sensor and determine the blood flow information through the lumen and communicate the blood flow information to the console.

Also disclosed is a method of measuring a blood flow through a vessel including, placing a prosthesis within the vessel, the prosthesis defining a lumen and including a graft coupled to an outer surface thereof, and a triboelectric nano-generator (TENG) coupled to an inner surface, moving the TENG relative to the prosthesis, generating a voltage output, transmitting the voltage output to a console, and determining a blood flow information through the lumen of the prosthesis.

In some embodiments, the step of moving the TENG further includes rotating the TENG relative to the prosthesis about a central longitudinal axis.

In some embodiments, the step of moving the TENG further includes expanding and contracting the TENG along an axis perpendicular to a central longitudinal axis of the prosthesis.

In some embodiments, the step of moving the TENG further includes moving the TENG back and forth, relative to the prosthesis, along an axis extending parallel to a central longitudinal axis of the prosthesis.

In some embodiments, the TENG includes a first material including one of polytetrafluoroethylene (PTFE), expanded polytetrafluoroethylene (ePTFE), or Kapton, and the graft includes a second material including one or more of polycarbonate, nylon, low density polyethylene (LDPE), or high density polyethylene (HDPE).

In some embodiments, the second material includes a difference in charge affinity (nC/J) of at least +/−40 nC/J relative to the first material.

In some embodiments, the TENG is coupled to the prosthesis at the upstream end and the downstream end to define an air gap between an outer surface of the TENG and an inner surface of the prosthesis.

In some embodiments, the prosthesis includes a third material including one of a plastic, polymer, nylon, metal, alloy, or Nitinol.

In some embodiments, transmitting the voltage output to a console further includes transmitting the voltage output by wired communication to the console coupled with the prosthesis.

In some embodiments, transmitting the voltage output to a console further includes transmitting the voltage output by wireless communication to the console coupled with an external computing device.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a restenosis detection system including a prosthesis having a triboelectric nano-generator (TENG) and an external computing device, in accordance with embodiments disclosed herein.

FIGS. 2A-2B show cross-sectional views of a rotational TENG prosthesis, in accordance with embodiments disclosed herein.

FIGS. 3A-3B show cross-sectional views of a radial TENG prosthesis, in accordance with embodiments disclosed herein.

FIGS. 4A-4B show cross-sectional views of a linear TENG prosthesis, in accordance with embodiments disclosed herein.

FIG. 5 shows a schematic view of a restenosis detection system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

In the following description, certain terminology is used to describe aspects of the invention. For example, in certain situations, the term “logic” is representative of hardware, firmware or software that is configured to perform one or more functions. As hardware, logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage. As used herein, one or more logic engines can use predetermined rule sets, machine learning algorithms, artificial intelligence (AI), neural networks, or the like to perform one or more functions, as described herein.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

A “message” generally refers to information transmitted in one or more electrical signals that collectively represent electrically stored data in a prescribed format. Each message may be in the form of one or more packets, frames, HTTP-based transmissions, or any other series of bits having the prescribed format.

The term “computerized” generally represents that any corresponding operations are conducted by hardware in combination with software and/or firmware.

To assist in the description of embodiments described herein, a longitudinal axis extends substantially parallel to an axial length of the prosthesis, substantially parallel to direction of flow through prosthesis. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

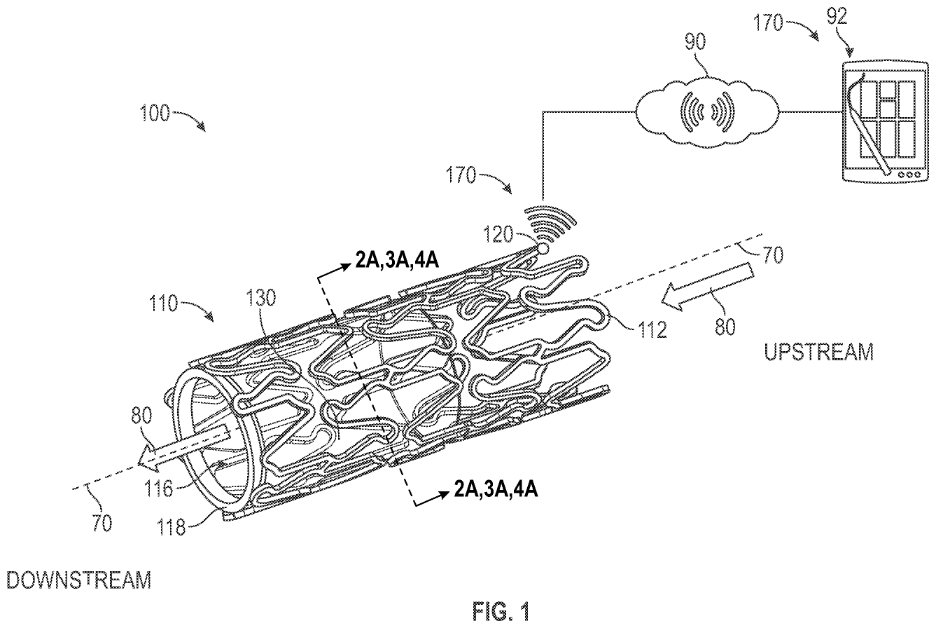

FIG. 1 shows a restenosis detection system (“system”) 100 generally including an intravascular prosthesis, or “stent,” 110 communicatively coupled with an external computing device 92 by way of a wireless communication antenna 120, such as WiFi, bluetooth, Near Field Communication (NFC), radio frequency (RF), infrared (IR), near infrared (NIR), acoustic, ultrasonic communication, or similar suitable means. In an embodiment, the computing device 92 can be handheld device, “smart” phone, tablet, laptop, computer, server, or the like. In an embodiment, the computing device 92 can be communicatively coupled with the stent 110 either directly, or indirectly by way of a centralized or decentralized network 90. Exemplary networks 90 can include one or more local area networks (LAN), intranets, internet, “cloud” based networks, servers, hospital networks, electronic health record (EHR) networks, or the like.

In an embodiment, the stent 110 can include a selectively expandable frame 112, defining a lumen 116, and formed of a plastic, polymer, metal, alloy, composite, stainless steel, Nitinol, polycarbonate, medical grade nylon, low-density polyethylene (LDPE), high-density polyethylene (HDPE), or similar suitable material. The frame 112 can be selectively expandable between a first diameter and a second diameter, the second diameter is larger than the first diameter. In use, the stent 110 in the first diameter configuration can be advanced through the vasculature to a target location, for example an occluded, or partially occluded area of vessel caused by atherosclerosis, or the like. Once at the target location, the stent 110 can be expanded to the second diameter configuration to restore patency to the vessel.

In an embodiment, the stent 110 can further include a graft 114 disposed on an outer surface of the frame 112. The graft 114 can be a permeable, impermeable, or selectively permeable membrane formed of a plastic, polymer, polycarbonate, nylon, low-density polyethylene (LDPE), high-density polyethylene (HDPE), polytetrafluoroethylene (PTFE), expanded polytetrafluoroethylene (ePTFE), polyimide films, poly (4,4′-oxydiphenylene-pyromellitimide) (“Kapton”), or the like. In an embodiment, the graft 114 can be flexible and can elastically or plastically deform between the first diameter and the second diameter of the frame 112.

As discussed herein, restenosis can occur where plaques, or similar deposits, can form on an inner surface of the stent 110, reducing the cross-section area of the lumen 116 and thereby reducing the patency of the vessel. Detecting the onset and progression of such deposits can provide an early indication for the onset of restenosis.

In an embodiment, the stent 110 can further include a triboelectric nano-generator (TENG) 130. The TENG 130 can comprise a tubular structure slidably and/or rotatably engaged with in inner surface of the frame 112. In an embodiment, the stent 110 can further include a collar 118 disposed at either an upstream or downstream end of the stent 110 and configured to retain the TENG 130 within the lumen 116 of the stent 110. The collar 118 can be secured in place using adhesive, bonding, welding, ultrasonic welding, mechanically bonded, heat fusion, stitched, or the like. In an embodiment, one or more of the TENG 130, the frame 112, and the graft 114 can be secured together at one or both of the upstream and downstream ends using adhesive, bonding, welding, ultrasonic welding, mechanically bonded, heat fusion, stitched, or the like.

In an embodiment, the TENG 130 is configured to convert mechanical energy from the flow of blood through the stent lumen 116 to electrical energy. Changes in mechanical energy can result in changes in electrical energy which in turn can be communicated to a console 170 and/or an external computing device 92 by way of an antenna 120. In an embodiment, the antenna 120 can be a printed antenna. In an embodiment, the console 170 can be printed circuitry, “system on a chip,” or similar device and can be disposed on the stent 110 or coupled with the external computing device 92, as described in more detail herein.

In an embodiment, the console 170 is configured to receive a signal, or voltage output from the TENG 130 and determine information about a blood flow 80 through the stent lumen 116. In an embodiment, the console 170 can be configured to determine an Oscillatory Shear Index (OSI) or Relative Residence Time (RRT) for a blood flow 80 through the stent lumen 116. As used herein, the OSI is a metric which characterizes whether the wall shear stress (WSS) vector is aligned with the time-average wall shear stress (TAWSS) vector throughout the cardiac cycle and quantifies deviations in the direction of the WSS vector throughout the cardiac cycle. As used herein, the Relative Residence Time (RRT) is a marker of disturbed blood flow, marked by low magnitude and high OSI.

In an embodiment, a blood flow 80 through the stent lumen 116 can result in one or more of a rotational, a radial, or a linear movement of the TENG 130 within the stent 110. It will be appreciated that other configurations and modes of TENG 130 are also contemplated, including but not limited to vertical contact-separation mode, lateral sliding mode, single-electrode mode, freestanding triboelectric-layer mode, or the like. The movement of the TENG 130 can then be converted to electrical signals and communicated to the external computing device 92, as described herein.

In an embodiment, sealing the upstream and downstream ends of the TENG 130 relative to the frame 112/graft 114 assembly can maintain an air gap 132 therebetween. The air gap 132 can allow the TENG 130 to move relative to the frame 112/graft 114 assembly in one or more of a rotational, radial, or linear movement, as described herein.

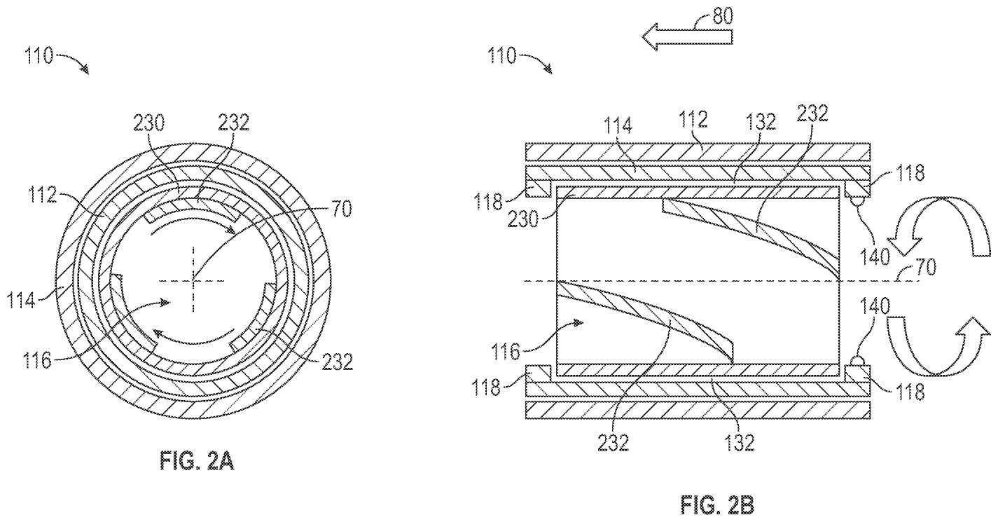

FIGS. 2A-2B show an exemplary rotational TENG 230. FIG. 2A shows a lateral cross-sectional view, extending perpendicular to a central axis 70 of the stent 110. FIG. 2B shows a longitudinal cross-sectional view, extending parallel to a central axis 70 of the stent 110.

As noted, the rotational TENG 230 can be rotatably and slidably engaged with an interior surface of the frame 112 such that the rotational TENG 230 can rotate freely about a central axis 70 of the stent 110. The TENG 230 can further include fins 232 disposed on an inner surface of the TENG. The fins 230 can extend over a portion of the inner surface of the TENG 230 and can be angled relative to the direction of blood flow 80 through the stent 110, i.e. at an angle to the central axis 70. As the blood flow passes through the stent lumen 116, a portion of the fluid can impinge on the fins 232 and cause the TENG 230 to rotate about the central axis 70, either clockwise or counterclockwise, relative to one or both of the frame 112 and graft 114.

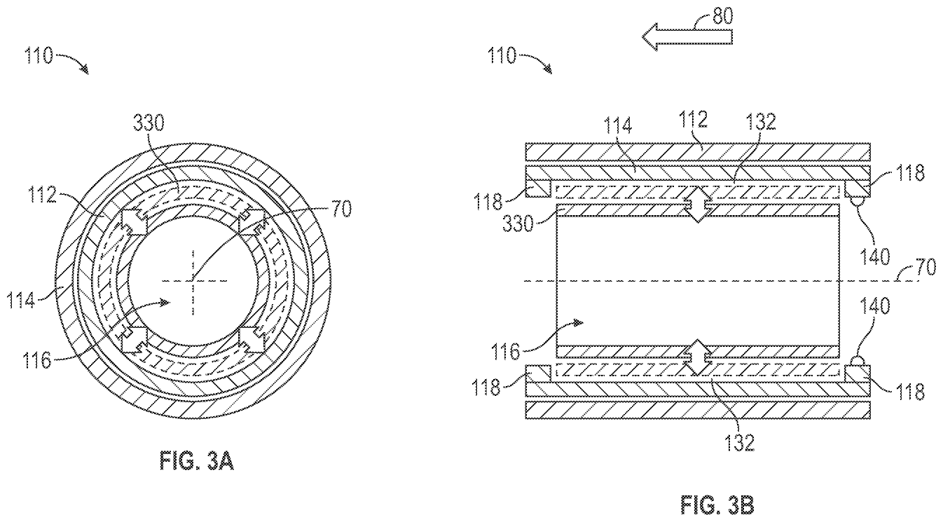

FIGS. 3A-3B show an exemplary radial TENG 330. FIG. 3A shows a lateral cross-sectional view, extending perpendicular to a central axis 70 of the stent 110. FIG. 3B shows a longitudinal cross-sectional view, extending parallel to a central axis 70 of the stent 110. The radial TENG 330 can be slidably engaged with an interior surface of the frame 112 such that the radial TENG 330 can expand and contract within the lumen 116 of the stent 110 relative to one or both of the frame 112 and graft 114, which would remain relatively stationary. For example, with each systolic cycle of the blood flow through the stent lumen 116, the radial TENG 330 can expand and contract along an axis extending perpendicular to the central longitudinal axis 70, causing the TENG 330 to move relative to one or both of the frame 112 and graft 114.

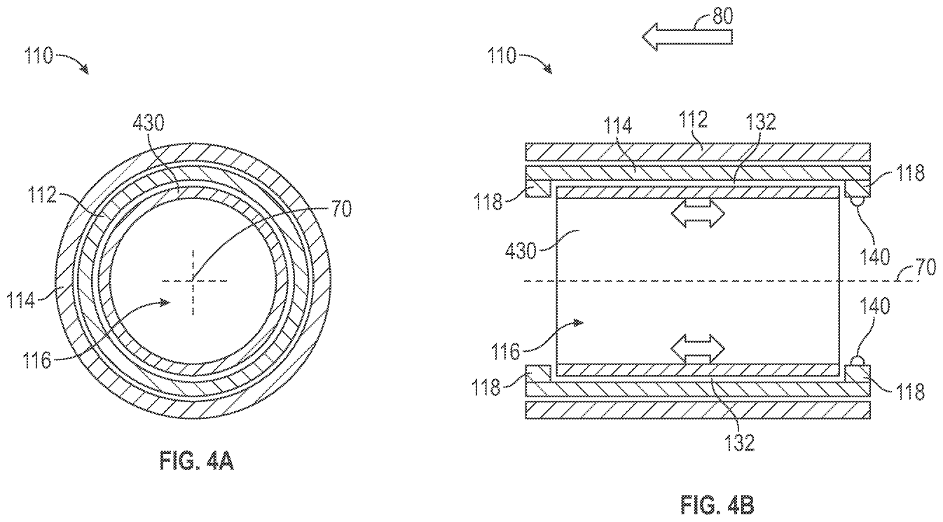

FIGS. 4A-4B show an exemplary linear TENG 430. FIG. 4A shows a lateral cross-sectional view, extending perpendicular to a central axis 70 of the stent 110. FIG. 4B shows a longitudinal cross-sectional view, extending parallel to a central axis 70 of the stent 110. The linear TENG 430 can be slidably engaged with an interior surface of the frame 112 along an axis extending parallel to the central longitudinal axis 70. The frictional force between the flow of blood through the TENG 430 can cause the TENG 430 to move back and forth along the longitudinal axis. As such, with each systolic cycle of blood flow through the stent lumen 116, the linear TENG 430 can move back and forth relative to one or both of the frame 112 and graft 114.

In an embodiment, the TENG 130 can be formed of a first material, and one or both of the stent frame 112 and the graft 114 can be formed of a second material, different from the first material. In an embodiment, the frame 112 can be formed of a third material different from both the first material and the second material. In an embodiment, one or more of the first material, second material, and the third material can be formed of a plastic, polymer, metal, alloy, composite, stainless steel, Nitinol, polycarbonate, nylon, low-density polyethylene (LDPE), high-density polyethylene (HDPE), or similar suitable material.

In an embodiment, the first material can provide one of a positive charge affinity or a negative charge affinity. The second material and/or the third material can provide an opposite charge affinity from that of the first material. In an embodiment, the second material and/or the third material can provide a neutral charge affinity relative to the first material. In an embodiment, the second material and/or the third material can provide a difference in charge affinity of +/−40 nC/J (nano ampsec/wattsec of friction) however greater or lesser differences in charge affinity are also contemplated.

Table 1 below provides some exemplary materials and associated charge affinities in (nC/J) for one or more of the first material, second material, and third material. As will be appreciated, these materials are exemplary and not intended to be limiting in any way.

TABLE 1
Exemplary materials and associated charge affinities in (nC/J) for
one or more of the first material, second material, and third material

| | Charge |
|---|---|
| | Affinity |
| Insulator | (nC/J) |
| Polyurethane foam | +60 |
| Sorbothane | +58 |
| Solid polyurethane | +40 |
| Nylon, dry skin | +30 |
| Nylon (Nylatron) | +28 |
| Nitrile rubber | +3 |
| Polycarbonate | −5 |
| Acrylic | −10 |
| Epoxy | −32 |
| Styrene-butadiene rubber | −35 |
| Polyethylene terephthalate (PET; Mylar) solid | −40 |
| Ethylene-vinyl acetate (EVA) rubber | −55 |
| Gum Rubber | −60 |
| Polystyrene | −70 |
| Polyimide | −70 |
| Silicones | −72 |
| Vinyl: flexible | −75 |
| Low-density polyethylene (LDPE) | −90 |
| High-density polyethylene (HDPE) | −90 |
| Polypropylene | −90 |
| Ultra-high-molecular-weight polyethylene (UHMWPE) | −95 |
| Polychloroprene | −98 |
| Poly-vinyl chloride (PVC; rigid vinyl) | −100 |
| Latex (natural) rubber | −105 |
| Fluoroelastomers (FKM; Viton, filled) | −117 |
| Epichlorohydrin rubber | −118 |
| Santoprene rubber | −120 |
| Hypalon rubber, filled | −130 |
| Butyl rubber, filled | −135 |
| Ethylene propylene diene monomer (EDPM) rubber, | −140 |
| filled | |
| Polytetrafluoroethylene (PTFE; Teflon) | −190 |

In an embodiment, the first material can be one of PTFE, ePTFE, a polyimide film, or poly (4,4′-oxydiphenylene-pyromellitimide) (“Kapton”), and the second material can be one or more of polycarbonate, nylon, LDPE, or HDPE and can be coupled to the antenna 120 by way of a conductive material. In an embodiment, the third material can be a metal, alloy, stainless steel, Nitinol, or the like. In an embodiment, the first material can be one or more of polycarbonate, nylon, LDPE, or HDPE and the second material can be one of PTFE, ePTFE, a polyimide film, or poly (4,4′-oxydiphenylene-pyromellitimide) (“Kapton”), and can be coupled to the antenna 120 by way of a conductive material. To note, the greater the difference in charge affinity (nC/J) between the first material and the second material, the stronger the voltage output is created by the TENG 130.

In an embodiment, the signal voltage output can be communicated to a small onboard console 170, e.g. located with the antenna 120, and can be analyzed locally on the stent 110. The blood flow information can then be communicated wirelessly to an external computing device 92.

In an embodiment, the signal voltage output can be communicated to the antenna 120 and can communicated wirelessly to an external computing device 92. The voltage signal output can then be analyzed by the console 170 coupled with the external computing device 92 to determine blood flow information.

In an embodiment, the stent 110 can further include a blood flow sensor 140 that is disposed either upstream or downstream of the TENG 130 and configured to determine a flow rate through the stent lumen 116. The blood flow sensor 140 can use one or more modalities to determine a blood flow through the stent lumen 116. Exemplary modalities can include mechanical-electrical, optical, acoustic, electromagnetic, radio frequency (RF), or the like. In an embodiment, the TENG 130 can provide power to the blood flow sensor 140 to determine a blood flow rate and communicate the blood flow information to the console 170 and/or external computing device 92, as described herein.

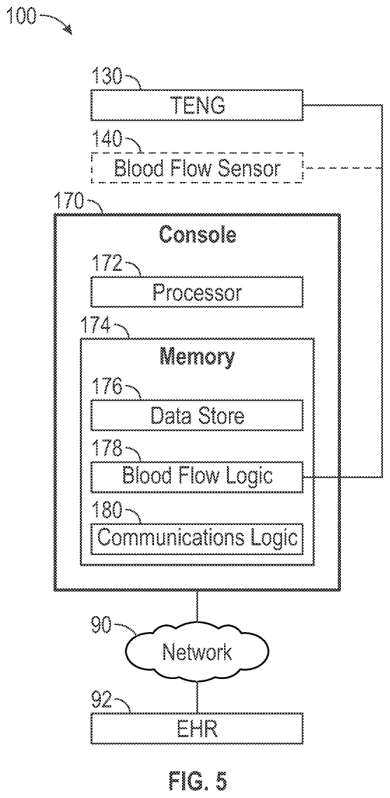

FIG. 5 provides a schematic view of the system 100 including the console 170. As noted, the console 170 can be disposed on one or both of the stent 110 (e.g. at antenna 120), or an external computing device 92 which is communicatively coupled with the stent 110 by way of the antenna 120. The console 170 can generally include one or more of a processor 172, and memory 174, including a data store 176 and one or more logic engines, such as a blood flow logic 178, and a communications logic 180. The console 170 can be communicatively coupled with the TENG 130 by either wired or wirelessly, as described herein.

In an embodiment, the voltage output from the TENG 130 can be received by the console 170 and the blood flow information logic 178 can determine a blood flow rate through the stent lumen 116. In an embodiment, the blood flow logic can use one or more of predetermined rule sets, machine learning (ML), artificial intelligence (AI), neural networks, or similar machine learning schema to determine blood flow information from the voltage output from the TENG 130. The blood flow information can either be stored locally on the data store 176 and/or can be communicated to an external computing device 92, e.g. an electronic health record (EHR) server, “cloud” server, or the like for further analysis.

Advantageously, the blood flow information from a patient can be monitored remotely and continuously in real-time to determine an onset of restenosis, a rate of progression of restenosis, or any abnormalities in the blood flow information which may indicate restenosis. Further, the system 100 does not require a power source to be located on the stent 110 since the power is generated by the TENG 130. Advantageously, this provides for a smaller device, with an extended lifespan, and can be placed intravascularly.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A system for detecting restenosis of a vessel, comprising:
a prosthesis disposed within the vessel and defining a lumen, the prosthesis comprising:
a graft disposed on an outer surface of the prosthesis; and
a triboelectric nano-generator (TENG) rotatably coupled to an inner surface of the prosthesis, the TENG configured to rotate relative to the prosthesis to generate a voltage output; and
a console communicatively coupled to the prosthesis and configured to receive and analyze the voltage output and determine a blood flow information including determining a flow rate through the lumen of the prosthesis.

2. (canceled)

3. The system according to claim 1, wherein the TENG is slidably coupled to the prosthesis and detects a change in blood flow through the lumen by a change in radial outward and inward movement of the TENG relative to the prosthesis.

4. The system according to claim 1, wherein the TENG is slidably coupled to the prosthesis and detects a change in blood flow through the lumen by a change in back and forth linear movement of the TENG, parallel to a direction of blood flow, relative to the prosthesis.

5. The system according to claim 1 4, wherein the TENG includes a first material, the graft includes a second material, the first material includes one of polytetrafluoroethylene (PTFE), expanded polytetrafluoroethylene (ePTFE), or Kapton, and the second material includes one or more of polycarbonate, nylon, low density polyethylene (LDPE), or high density polyethylene (HDPE).

6. The system according to claim 5, wherein the second material includes a difference in charge affinity (nC/J) of at least +/−40 nC/J relative to the first material.

7. The system according to claim 1, wherein the prosthesis includes a frame formed of a third material including one of a plastic, polymer, nylon, metal, alloy, or Nitinol.

8. The system according to claim 1 4, wherein the TENG includes a first material, the graft includes a second material, and the prosthesis further includes a frame formed of a third material, the first material, second material and third material being the same or different.

9. The system according to claim 8, wherein the first material may be selected from one or more of a plastic, polymer, polycarbonate, nylon, low-density polyethylene (LDPE), high-density polyethylene (HDPE), polytetrafluoroethylene (PTFE), expanded polytetrafluoroethylene (ePTFE), polyimide films, and poly (4,4′-oxydiphenylene-pyromellitimide) (“Kapton”).

10. The system according to claim 8, wherein the second material may be selected from one or more of a plastic, polymer, polycarbonate, nylon, low-density polyethylene (LDPE), high-density polyethylene (HDPE), polytetrafluoroethylene (PTFE), expanded polytetrafluoroethylene (ePTFE), polyimide films, poly (4,4′-oxydiphenylene-pyromellitimide) (“Kapton”), metal, alloy, composite, stainless steel, and Nitinol.

11. The system according to claim 8, wherein the second material may be selected from one or more of a plastic, polymer, polycarbonate, nylon, low-density polyethylene (LDPE), high-density polyethylene (HDPE), polytetrafluoroethylene (PTFE), expanded polytetrafluoroethylene (ePTFE), polyimide films, and poly (4,4′-oxydiphenylene-pyromellitimide) (“Kapton”).

12. The system according to claim 1, wherein the prosthesis is configured to transition between a first configuration having a first diameter and a second configuration having a second diameter, the second diameter being larger than the first diameter.

13. The system according to claim 1, wherein the prosthesis further includes an antenna configured to communicatively couple the prosthesis to an external computing device.

14. The system according to claim 13, wherein the console is disposed on the prosthesis and is configured to determine the blood flow information and communicate the blood flow information to the external computing device.

15. The system according to claim 13, wherein the console is disposed on the external computing device and the antenna communicates voltage output from the TENG to the console to determine the blood flow information.

16. The system according to claim 13, wherein the prosthesis further includes a blood flow sensor configured to receive a voltage output from the TENG to power the blood flow sensor and determine the blood flow information through the lumen and communicate the blood flow information to the console.

17. A method of measuring a blood flow through a vessel, comprising:
placing a prosthesis within the vessel, the prosthesis defining a lumen and including
a graft coupled to an outer surface thereof, and a triboelectric nano-generator (TENG) rotatably coupled to an inner surface;
rotating the TENG relative to the prosthesis about a central longitudinal axis;
generating a voltage output;
transmitting the voltage output to a console; and
determining a blood flow information including determining a flow rate through the lumen of the prosthesis.

18. (canceled)

19. The method according to claim 17, further including expanding and contracting the TENG along an axis perpendicular to a central longitudinal axis of the prosthesis to generate a voltage output and determine a blood flow information.

20. The method according to claim 17, further including moving the TENG back and forth, relative to the prosthesis, along an axis extending parallel to a central longitudinal axis of the prosthesis to generate a voltage output and determine a blood flow information.

21. The method according to claim 17, wherein the TENG includes a first material including one of polytetrafluoroethylene (PTFE), expanded polytetrafluoroethylene (ePTFE), or Kapton, and the graft includes a second material including one or more of polycarbonate, nylon, low density polyethylene (LDPE), or high density polyethylene (HDPE).

22. The method according to claim 21, wherein the second material includes a difference in charge affinity (nC/J) of at least +/−40 nC/J relative to the first material.

23. The method according to claim 17, wherein the TENG is coupled to the prosthesis at the upstream end and the downstream end to define an air gap between an outer surface of the TENG and an inner surface of the prosthesis.

24. The method according to claim 17, wherein the prosthesis includes a third material including one of a plastic, polymer, nylon, metal, alloy, or Nitinol.

25. The method according to claim 17, wherein transmitting the voltage output to a console further includes transmitting the voltage output by wired communication to the console coupled with the prosthesis.

26. The method according to claim 17, wherein transmitting the voltage output to a console further includes transmitting the voltage output by wireless communication to the console coupled with an external computing device.
